(19) United States
(12) Patent Application Publication
(10) Pub. No.: US 2023/0241126 A1
(43) Pub. Date: Aug. 3, 2023

(54) COMPOSITIONS AND METHODS FOR DISRUPTION OF BIOFILMS USING FRACTIONATED HONEY

(71) Applicant: NDAL MFG INC, Monterey, CA (US)

(72) Inventors: Gavin Mark Gear, Monterey, CA (US); Roger Brett Edmonds, Auckland (NZ)

(21) Appl. No.: 18/101,048

(22) Filed: Jan. 24, 2023

Related U.S. Application Data
(60) Provisional application No. 63/304,209, filed on Jan. 28, 2022.

(51) Int. Cl. A61K 35/644; A61K 47/02; A61K 47/10; A61K 47/12; A61K 47/22; A61P 31/00

(52) U.S. Cl. CPC A61K 35/644; A61K 47/02; A61K 47/10; A61K 47/12; A61K 47/22; A61P 31/00

(57) ABSTRACT

A method of disrupting a biofilm in a closed water system, the method comprising: introducing to the closed water system a composition comprising a fractionated honey in an amount effective for disrupting a biofilm in the closed water system. A method of disrupting a biofilm caused by a bacteria or virus formed on an abiotic surface, the method comprising: applying to an abiotic surface a composition comprising a fractionated honey produced from Leptospermum scoparium in an amount effective for disrupting a biofilm formed on the abiotic surface.

### CROSS-REFERENCE TO RELATED APPLICATION

The application is a non-provisional application of U.S. Provisional Patent Application No. 63/304,209, filed Jan. 28, 2022, the contents of which is incorporated herein by reference in their entirety.

### BACKGROUND

### Field

Compositions and methods for disruption of biofilms caused by a pathogen including a bacterium, a virus or other causes using a fractionated honey.

### Background

There are a number of infections humans suffer from on a regular basis. Such infections are commonly caused by pathogens including viruses and bacteria. For example, respiratory infections including lung, nasal and/or sinus infections may be caused by one or more of rhinovirus, coronavirus, parainfluenza virus, respiratory syncytial virus, adenovirus, influenza viruses, enterovirus, metapneumovirus, coronavirus 229E, OC43, NL63 and HKU1, coronavirus COVID-19, MERS-CoV, SARS-CoV, influenza A & B viruses, parainfluenza type 1 & type 2, streptococcal, pneumococcal, staphylococcal, avian influenza H5, H7, H9 viruses, metapneumovirus, streptococci, pneumococci, Moraxella catarrhalis, staphylococci, Haemophilus influenzae, Moraxella catarrhalis, staphylococci, Staphylococcus aureus, Klebsiella pneumoniae Pseudomonas aeruginosa, Proteus mirabilis, Enterobacter, Staphylococcus epidermidis, Propionibacterium acnes, coagulase negative Staphylococcus, Streptococcus pneumoniae, Prevotella Streptococcus, Veillonella, gram negative bacilli and/or oropharyngeal anaerobic microorganisms. It is believed that many of the infections, and particularly chronic infections, caused by these viruses and/or bacteria or infectious, vasomotor, drug induced (eg, aspirin or nonsteroidal anti-inflammatory drug [NSAID]-induced) and atrophic rhinitis, are the result of biofilms forming on biotic surfaces, and specifically tissues surfaces within the respiratory tract. In addition, biofilms can form on non-living tissues or abiotic surfaces. For example, biofilms can form on metal or other non-living surfaces allowing pathogens to spread. For example, a biofilm may form on the surface of structures used in industrial closed water systems and contaminate the contents of the system. In other aspects, a biofilm may form on the surface of a medical implant or other foreign object introduced within the body and/or equipment used in a healthcare setting. Biofilms can therefore also be associated with infections within hospitalized patients and/or other patients in health care settings. A biofilm is essentially a cluster of bacteria held together by a mucus-like material that adheres the biofilm to the surface on which it forms. The production of biofilms is achieved through external signals followed by the activation of specific genes.

### SUMMARY

In one aspect, the disclosure is directed to a method of disrupting a biofilm in a closed water system, the method comprising: introducing to the closed water system a composition comprising a fractionated honey in an amount effective for disrupting a biofilm in the closed water system. In some aspects, the biofilm is formed on a surface within the closed water system. In other aspects, the biofilm is caused by a gram positive bacterium, a gram negative bacterium or a virus. In some aspects, the composition comprises fractionated honey in an amount of from 20% to 80% by weight of the composition. In some aspects, the composition comprises fractionated honey in an amount of 25% to 50% by weight of the composition. In still further aspects, the composition comprises fractionated honey in an amount of 50% to 75% by weight of the composition. In still further aspects, introducing the composition includes applying the composition and maintaining the composition in the closed water system for a predetermined period of time. In some aspects, the predetermined period of time may be 60 minutes or more. In some aspects, the composition may include fractionated honey in an amount of from 20% by weight to 30% by weight of the composition, and the predetermined period of time may be from 80 minutes to 100 minutes. The method may further include removing the composition from the closed water system once the biofilm is disrupted. In some aspects, removing may include flushing the composition from the closed water system.

In another aspect, the disclosure is directed to a method of disrupting a biofilm caused by a bacteria or virus formed on an abiotic surface, the method comprising: applying to an abiotic surface a composition comprising a fractionated honey produced from Leptospermum scoparium in an amount effective for disrupting a biofilm formed on the abiotic surface. In some aspects, the abiotic surface comprises a surface of a medical implant. In some aspects, the abiotic surface comprises a surface within a closed water system. In some aspects, the amount of fractionated honey is at least 20% by weight of the composition. In other aspects, the amount of fractionated honey is at least 50% by weight of the composition. In some aspects, the amount of fractionated honey is at least 75% by weight of the composition. In still further aspects, the amount of fractionated honey is 80% or less by weight of the composition. In some aspects, the composition is in the form of a spray.

The above summary does not include an exhaustive list of all aspects of the present disclosure. It is contemplated that the disclosure includes all compositions, methods, kits and/or systems that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence is intended to have any limiting effect on the scope of any claim elements. For purposes of clarity, not every component may be labeled in every figure. The figures are provided for the purposes of illustration and explanation and are not intended as a definition of the limits of the invention. In the figures:

FIG. 1 shows a representative confocal micrograph for a control in accordance with some embodiments of the technology described herein;

FIG. 2 shows a representative confocal micrograph for a control in accordance with some embodiments of the technology described herein;

FIG. 3 shows a representative confocal micrograph for a treatment in accordance with some embodiments of the technology described herein;

FIG. 4 shows a representative confocal micrograph for a treatment in accordance with some embodiments of the technology described herein;

FIG. 5 shows a representative confocal micrograph for a treatment in accordance with some embodiments of the technology described herein; and

FIG. 6 shows a representative confocal micrograph for a treatment in accordance with some embodiments of the technology described herein.

FIG. 7 shows a representative process flow for disrupting a biofilm.

### DETAILED DESCRIPTION

In this section we shall explain several preferred embodiments of this invention. Whenever the compositions, formulations, methods and other aspects of the embodiments are not expressly defined, the scope of the invention is not limited only to the disclosed embodiments, rather may encompass what is well known in the art. Also, while numerous details are set forth, it is understood that some embodiments of the invention may be practiced without these details. In other instances, well-known chemicals, formulations, ingredients and techniques have not been described in detail so as not to obscure the understanding of this description.

In one aspect, the instant invention is directed to a composition and method of using fractionated honey to disrupt biofilms formed on abiotic surfaces and/or in abiotic systems. The term “abiotic” is intended to refer to any material, surface, object, device or the like which is not derived from a living organism. In one aspect, the abiotic surface may be a surface that forms a part of, or is otherwise within, an industrial closed water system. Representatively, an industrial closed water system may be understood as referring to any process system performed in a closed water environment (e.g., a metal vat) with equipment designed and operated such that the product is not exposed to the room environment. Materials may be introduced to a closed system, but the addition is done in such a way to avoid exposure to the product in the room environment. Typically, closed water systems will reduce the risk of contamination since they are not open to the room environment. In some cases, however, an agent may inadvertently enter the system resulting in the formation of biofilms on the surfaces of the structures and/or equipment used in the system. Biofilms in these water systems are very disruptive to pharmaceutical companies that use such water systems for manufacturing. Currently toxic chemicals are used to remove biofilm contaminations followed by extreme measures eliminate these toxic chemicals from the water system before resuming production. The composition and method disclosed herein may therefore be used to remove and/or disrupt these biofilms formed on metal or other abiotic or inanimate surfaces without the need for toxic chemicals to remove them. For example, the composition may be used to disrupt a biofilm formed on the surface of structures used in closed water systems to prevent contamination of the contents of the system. In other aspects, the composition may be used to disrupt a biofilm formed on other abiotic surfaces, for example, a metal surface, a ceramic surface, a plastic surface, a rubber surface, a silicon surface, a polyvinyl surface, or any other abiotic or non-living surface. Representatively, in one aspect, the abiotic surface may be a metal surface of a medical implant which is implanted within the body or a medical device that is temporarily inserted into the body. Examples of abiotic surfaces and/or medical implants or devices having abiotic surfaces, may include, but are not limited to, a catheter, stent, a butterfly needle, a winged infusion set, a scalp vein set or any other device used to access a vein for drawing blood or giving medications, or any device the penetrates the skin or the body, to prevent infection to the user. In another aspect, the abiotic surface may be a surface of an artificial airway device upon which a biofilm may form, for example, the surface of an endotracheal tube, a tracheostomy tube, or the like. In other aspects, the invention may also be directed to the use of fractionated honey to treat or prevent persons from suffering from a condition such as an infection associated with a biofilm by disrupting the bacterial cell culture and inhibiting the bacterial biofilm. The fractionated honey is considered “fractionated” in that it is formed by taking Manuka honey and removing the DNA marker (pollen) from the honey. Manuka honey typically includes a number of markers that must be present for it to be considered an authentic Manuka honey. Among these markers is the DNA level from Manuka pollen, which is typically less than Cq 36 for Manuka honey. In the proposed fractionated honey, this DNA marker is removed such that the DNA level from Manuka pollen is essentially zero. The resulting fractionated honey maintains all the health benefits of Manuka honey without some of the side effects that may be associated with pollen, for example, allergic reactions. In addition, in some instances where the honey may be irradiated to achieve sterility, the removal of the pollen may have other benefits. For example, when honey is irradiated the pollen or contaminate may explode and become particulate matter in the honey, or a contaminate with potential to cause inflammation. Thus, the removal of pollen reduces the potential for the pollen to cause potential contaminates that may lead to undesirable inflammation in instances where the honey is irradiated, and in turn reduce the overall effectiveness of the honey.

While all honey is believed to have health benefits, Manuka honey includes unique antibacterial ingredients not found in other honeys believed to make it have superior anti-bacterial, anti-inflammatory and/or anti-viral properties to other types of honey. In particular, Manuka honey is a plant specific honey made by bees who collect nectar from New Zealand's native Leptospermum scoparium plant. As a result, Manuka honey contains a unique anti-bacterial ingredient not found in other honeys that is believed to make it an effective antibiotic and wound healer. In particular, Manuka honey naturally contains methylglyoxal (MGO), which is a chemical known to have antibacterial properties. In particular, MGO is a highly reactive compound which can readily react with cellular molecules. The chemical reactions between MGO and cellular molecules in the bacteria, damages molecules needed for bacterial viability. MGO is also an indicator of other active antibacterial or antiviral fractions within the fractionated honey and the precise mode of action of certain active fractions remains unknown, we see only the result of those active fractions. The higher the MGO content, the higher the grade of the Manuka honey. The fractionated honey disclosed herein maintains the MGO content without any allergy inducing pollen, thus resulting in an effective antibacterial and anti-inflammatory without any of the undesirable side effects sometimes associated with honey.

It is proposed herein that the MGO present in the proposed fractionated honey helps to treat and/or prevent diseases and/or infections by disrupting the bacterial cell structure and/or attachment of biofilm and/or cellular death. The disease and/or infections that may cause a biofilm and are suitable for treatment using the fractionated honey may be caused by one or more of a bacteria (e.g., gram-positive or gram-negative) or a virus.

In still further aspects, the proposed fractionated honey composition may be used as a prophylactic or preventative measure to prevent the user from developing or contracting any of the conditions disclosed herein. For example, the fractionated honey composition may provide immune support when administered prior to an infection or condition by improving sinus health and protecting the sinuses against particulate matter, bacteria, viruses, bed mites, pet dander, dust, pollen and/or other allergens. Representatively, honey, and particularly Manuka honey, has been found to efficiently inhibit influenza virus replication, which is related to its virucidal effects. Watanabe K, et al., Anti-influenza Viral Effects of Honey In Vitro: Potent High Activity of Manuka Honey, Arch Med Res. 2014 July; 45(5):359-365.

Representative diseases, infections, viruses and/or bacteria that are suitable for treatment or prevention using the fractionated honey disclosed herein may include, but are not limited to, adenoviruses, avian influenza H5, H7, and H9 viruses, coronaviruses 229E, OC43, NL63, and HKU1, SARS-CoV-2, COVID-19, MERS-CoV, SARS-CoV, enteroviruses, influenza viruses, influenza A and B viruses, H3N2 (an influenza A virus), metapneumoviruses, parainfluenza virus, parainfluenza viruses for example paramyxoviruses classified as types 1, 2, 3, and 4, respiratory syncytial virus, rhinovirus, nonallergic (e.g., nonallergic forms of perennial rhinitis including infectious, vasomotor, drug-induced and atrophic rhinitis) or allergic rhinitis, acute rhinitis, streptococcal, pneumococcal, staphylococcal infections, chronic rhinitis, rhinoscleroma, rhinosporidiosis, atrophic rhinitis, vasomotor rhinitis, sinusitis, haemophilus influenza, influenza, Moraxella catarrhalis, or staphylococci, parainfluenza, pneumococci, rhinovirus, streptococci, enterobacter, gram negative bacilli and/or oropharyngeal anaerobic microorganisms, Klebsiella pneumoniae, Proteus mirabilis, and Propionibacterium acnes, prevotella streptococcus, Pseudomonas aeruginosa, Staphylococcus aureus, Staphylococcus epidermidis, Coagulase negative staphylococcus, Streptococcus pneumonia and/or veillonella, Pneumocystis carinii, cytomegalovirus, methicillin-resistant Staphylococcus aureus (MRSA), Acinetobacter baumannii, chlamydophila pneumonia and/or Mycoplasma pneumoniae. In still further aspects, the diseases, infections, viruses and/or bacteria that are suitable for treatment or prevention using the fractionated honey disclosed herein may include, but are not limited to, lung conditions, asthma, cystic fibrosis, clostridium, campylobacter concisus biofilm (e.g., acid reflux), oesophageal bacterial biofilm and/or any other condition of the sinus.

For example, the diseases and/or infections may be caused by one or more bacterium and may include, but are not limited to, Staphylococcus aureus, Haemophilus influenza, Pseudomonas aeruginosa, Staphylococcus epidermidis, Propionibacterium acnes, coagulase negative Staphylococcus, Streptococcus pneumoniae, Prevotella Streptococcus, Veillonella, Proteus mirabilis, methicillin-resistant Staphylococcus aureus (MRSA) and/or Klebsiella pneumonia.

Diseases and/or infections that may cause a biofilm and are suitable for treatment using the fractionated honey disclosed herein may include, but are not limited to, those caused by viruses including, but not limited to, severe acute respiratory syndrome coronavirus (SARS-CoV), severe acute respiratory syndrome coronavirus 2 (SARS-CoV-2), Middle East respiratory syndrome coronavirus (MERS-CoV), respiratory syncytial virus (RSV), Rhinoviruses, Parainfluenza viruses, Influenza viruses, and/or Adenoviruses.

Representatively, it is proposed that the fractionated honey disclosed herein inhibits Staphylococcus aureus by interfering with the cell division process. Under optimal conditions, bacterial cells duplicate and segregate their chromosome, forming a proteinaceous ring (the septum) across the mid-cell, creating two still-joined daughter cells. The completion of cell division occurs when peptidoglycan (murein) hydrolases degrade the cell wall between the two daughter cells, allowing separation. Manuka honey has been shown to inhibit the activity (and not the expression) of murein hydrolase, causing a build-up of septated non-dividing cells.

In addition, it is proposed that where the infection is caused by Pseudomonas aeruginosa, the fractionated honey causes the Pseudomonas aeruginosa cells to lyse in its presence due to the reduction of a key structural protein. Representatively, in contrast to the mechanism observed in Staphylococcus aureus, studies have proposed an entirely different mechanism against Pseudomonas aeruginosa. Pseudomonas aeruginosa cells can tolerate higher concentrations of Manuka honey when compared to Staphylococcus aureus, with inhibitory concentrations causing the loss of cellular integrity, leading to extensive cell lysis and cell death. Pseudomonas aeruginosa modulates its structural integrity through the production of a key anchor protein: outer membrane protein F (OprF). This protein provides a vital link between the outer membrane and underlying peptidoglycan layer, ensuring cell envelope homeostasis and regular cell shape. Reduced OprF expression has been observed in populations treated with Manuka honey, and a concomitant increase in membrane blebbing and cell lysis has also been detected. The different mechanistic actions observed against Pseudomonas aeruginosa (compared to Staphylococcus aureus aureus) highlights the potential for multiple modes of action, and multiple inhibitory compounds in Manuka honey and, in turn, the fractionated honey disclosed herein.

Moreover, exposure to Manuka honey has been shown to have other effects against a range of organisms. Against Pseudomonas aeruginosa, manuka honey suppresses the class I master regulators (FleQ and FliA), inhibiting the regulatory cascade required for flagellum production and leading to a significant reduction in flagellated cells. This observation is of clinical significance as adhesion and cellular motility are required for invasive virulence. Invasive virulence is problematic, as it allows the dissemination of cells through the bloodstream (bacteremia) to internal organs, which can prove fatal; therefore, the potential to reduce this process is highly valuable. The ability of Pseudomonas aeruginosa to sequester iron from a host may also be prohibited through Manuka honey treatment, following the observation of reduced siderophore production in treated samples. Sub-inhibitory concentrations are shown to inhibit cellular binding with fibronectin through the loss of two streptococcal surface proteins, SoF and SfbI. In wound infections, high concentrations of fibronectin are observed; therefore the inability of Streptococcus pyogenes to bind to the host may impact on its pathogenicity.

In addition to the studies into Staphylococcus aureus, Pseudomonas aeruginosa, and Streptococcus pyogenes, a study into the global action of Manuka honey on Escherichia coli demonstrated that following exposure to Manuka honey, 2% of the genes were up-regulated, while 1% were down-regulated by twofold or more. Up-regulation appears to occur across genes involved in stress response; those genes down-regulated are thought to encode products involved in protein synthesis. Conversely, down-regulation (16-fold) of a universal stress protein A (UspA) in Staphylococcus aureus cells treated with honey was observed. Another study has shown large-scale down-regulation of critical virulence genes (enterotoxins, fibronectin-binding proteins, hemolysins, and lipases), with concomitant reductions in global regulators and quorum-sensing genes. These mechanistic effects, both lethal and non-lethal, are a testament to the inhibitory efficacy of Manuka honey (and the fractionated honey disclosed herein) and confirm its broad spectrum of effects. In addition to these inhibitory effects, the fractionated honey composition disclosed herein may reduce virulence, motility, and biofilm formation.

The fractionated honey composition disclosed herein may include additional improvements over typical honey formulations including an increased water activity level and/or decreased acidity, while still preventing the growth of harmful bacteria and preventing fermentation. By way of background, a carrier is required to deliver fractionated honey to abiotic and/or biotic systems. In some aspects, the carrier may be water. When the water content in a honey formulation is increased, however, honey is susceptible to fermentation, particularly at a water content of 19% and above. When water content and/or activity is lowered, the potential for fermentation is eliminated. In addition, increasing the acidity to pH levels of 4.6 or lower prevents the growth of harmful bacteria. For example, reducing the water activity of a food to below 0.85 (e.g., by adding a sugar or salt) and/or acidifying a food to a pH level of 4.6 or lower (e.g., by adding vinegar or lemon juice) will prevent harmful bacteria and help prevent fermentation. Alternatively, a chemical preservative can be added to protect the honey from fermenting. Reducing the water activity, increasing the acidity and/or the addition of chemical preservatives to the fractionated honey formulation, however, may not be desirable in the formulations proposed herein for disrupting biofilms.

Representatively, in the proposed fractionated honey formulation for disrupting biofilms disclosed herein, the water activity is maintained at a range greater than 0.85, for example, a range of from about 0.86 to about 1, for example, a range of from about 0.89 to about 0.9. In still further aspects the fractionated honey formulation may have a water content greater than 19% by weight. In addition, the pH range of the proposed fractionated honey formulation is maintained between about 3 to about 4, for example, a pH of about 3.2 to about 3.6, for example, a pH of around 3.4 to 3.5, or about 3.44. The reduced acidity and/or increased water activity of the proposed fractionated honey formulation, as compared to other formulations including honey, results in a formulation and/or composition which may be gentler, less irritating and more effective when used in biotic and/or abiotic systems as proposed herein.

The fermentation and/or bacterial growth is instead controlled by using a mixture of citric acid and ascorbic acid (e.g., vitamin C) which blocks access of the honey to the water. When the citric and ascorbic acid mixture is added to the water and absorbed into the water cells, the honey is blocked from accessing the water, thereby preventing fermentation and/or colony formation. The citric and ascorbic acid mixture therefore acts as a natural preservative for the formulation and/or water itself so that the fractionated honey formulation can maintain a low acidity and increased water activity, and without the need for sugars, salts or chemical preservatives, all of which can damage aspects of abiotic and/or biotic systems.

In one aspect, the composition including the fractionated honey may include a synergistic combination of one or more of a water, fractionated medical grade Manuka honey, vegetable Glycerin, salt, and/or a mixture including citric acid and ascorbic acid (e.g., vitamin C), that when combined, have an effect greater than the sum of theft separate effects at treating a human suffering from an infection (e.g., lung, nasal, sinus, etc), for example, an infection caused by one or more of Staphylococcus aureus, Haemophilus influenza, or Pseudomonas aeruginosa, Staphylococcus epidermidis, Propionibacterium acnes, coagulase negative Staphylococcus, Streptococcus pneumoniae, Prevotella Streptococcus, Veillonella, Proteus mirabilis, and/or Klebsiella pneumonia.

In one embodiment, the composition balances one or more of a water, fractionated medical grade Manuka honey, vegetable Glycerin, salt, and/or a mixture including citric acid and ascorbic acid (e.g., vitamin C) in amounts sufficient to effectively treat a human suffering from an infection (e.g., lung, nasal, sinus, etc), for example, an infection caused by one or more of Staphylococcus aureus, Haemophilus influenza, or Pseudomonas aeruginosa, Staphylococcus epidermidis, Propionibacterium acnes, coagulase negative Staphylococcus, Streptococcus pneumoniae, Prevotella Streptococcus, Veillonella, Proteus mirabilis, and/or Klebsiella pneumonia.

Representatively, in one aspect, the fractionated honey composition may be a composition including, among other ingredients, water. Water functions as a carrier or delivery mechanism of the honey. It is therefore desirable for the water content to be any amount that effectively delivers the honey to the desired tissue. For example, in the case where the formulation is delivered in the form of a nasal spray, the water may be in an amount that allows for a spray pump to deliver the formulation to the furthest sinus pockets. The water content should, however, remain low enough so as not to promote bacterial growth. The water may be a filtered water present in an amount of at least about 50 weight percent (wt %) of the composition or more, and in some cases 82 weight percent or less. In another aspect, the water may be considered present in a ratio of one.

In another aspect, the fractionated honey composition may be a composition including, among other ingredients, a fractionated honey. For example, the fractionated honey may be a Manuka honey that is processed to remove the DNA marker associated with Manuka pollen while still maintaining a desired concentration of the MGO antibacterial component found in the honey, as previously discussed. For example, the fractionated honey may include an MGO content of, for example, at least 83 mg/kg MGO, at least 115 mg/kg, at least 263 mg/kg, at least 400 mg/kg, at least 573 mg/kg, and more preferably at least 600 mg/kg or at least 700 mg/kg or more, for example, 800 mg/kg, 900 mg/kg or 1000 mg/kg. The fractionated honey may be included in the composition in an amount found effective for treating infections. For example, the fractionated honey may be included in an amount of at least 20 wt % of the composition. In another aspect, the fractionated honey may be considered present in a ratio of 1.41.

It should be understood that there is a direct relationship between the MGO content and the antibacterial and antiviral effect of honey. As previously discussed, typically a bacterial biofilm forms on a viral infection. The virus kills tissue, creating a breeding ground for the bacteria. An MGO 600 content is believed to kill the virus and the biofilm. It is further believed that an MGO content of more than 600, for example 800 or more, may kill the virus and the biofilm. One representative study in which the fractionated honey composition disclosed herein was found to exhibit virucidal activity when tested against SARS-CoV-2 will be disclosed in more detail in reference to Example 9.

In one aspect, the fractionated honey composition may be a composition including, among other ingredients, a glycerin. For example, the glycerin may be a vegetable glycerin. Glycerin is a tissue moisturizer and may be included in the composition to promote water absorption into tissues and for a soothing effect on the tissue. The glycerin may be included in the composition in an amount suitable for having a moisturizing and/or soothing effect on the tissue and/or found effective for treating infections. For example, the glycerin may be included in the composition in an amount of about 10% or less of the composition. In another aspect, the glycerin may be considered present in a ratio of 1.26.

In another aspect, the fractionated honey composition may be a composition including, among other ingredients, a salt. For example, the salt may be a pharmaceutical grade salt. The salt may be included in the composition in an amount found effective for treating infections. Salt may be included in the formulation to inhibit planktonic bacteria, open cells and/or to serve as a flushing agent when in solution with water. For example, the salt may be included in the composition in an amount of about 5% or less of the composition. In another aspect, the salt may be considered present in a ratio of 1. In another aspect, the fractionated honey composition may be a composition including, among other ingredients, a mixture of citric acid and ascorbic acid. For example, the ascorbic acid may be vitamin C. The mixture may further include an amount of water, for example, from 40 wt % to 50 wt % water. The mixture of citric acid, ascorbic acid and water may be referred to herein as the “acid mixture”. The acid mixture may be included in an amount and/or ratio found effective for blocking the access of the fractionated honey to water molecules and therefore prevent fermentation and/or bacterial growth. For example, the acid mixture may be included in the composition in an amount of about 1 wt % of the composition or less. In another aspect, the acid mixture may be considered present in a ratio of 1. Other ingredients or agents included in the composition that may not be specifically discussed above are included and described in reference to the exemplary formulations set forth below. In addition, it should further be understood that although the ingredients and/or agents described herein are categorized according to a single function, many have multiple functions and therefore may be understood to be included under other functional categories than those listed herein.

The following specific examples set forth exemplary compositions that may be administered to a subject and/or otherwise used to disrupt a biofilm formed on an inanimate object and/or within a closed water system. The ingredient amounts disclosed in the following example are in effective amounts suitable for disruption of a biofilm formed on an inanimate objection, living tissue and/or prevention or treatment of an infection (e.g., lung, nasal, sinus, etc) caused by one or more of the previously discussed viruses, bacteria, or other conditions, including, but not limited to, Staphylococcus aureus, Haemophilus influenza, or Pseudomonas aeruginosa, Staphylococcus epidermidis, Propionibacterium acnes, coagulase negative Staphylococcus, Streptococcus pneumoniae, Prevotella Streptococcus, Veillonella, Proteus mirabilis, Klebsiella pneumoniae, Pneumocystis carinii, cytomegalovirus, methicillin-resistant Staphylococcus aureus (MRSA), Acinetobacter baumannii, chlamydophila pneumonia and/or Mycoplasma pneumoniae and/or otherwise improving the condition of the area to which the composition is applied. In one embodiment, the composition may have the following exemplary formulations:

### Example 1

| | EFFECTIVE AMOUNT (weight % of |
|---|---|
| INGREDIENT | the composition) |
| Filtered Water | 50 wt % or more |
| Fractionated Manuka Honey | 20 wt % or more |
| Vegetable Glycerin | 5 wt % or less |
| Pharmaceutical Grade Salt | 1 wt % or less |
| Acid Mixture | 1 wt % or less |

### Example 2

| | EFFECTIVE AMOUNT (weight % of |
|---|---|
| INGREDIENT | the composition) |
| Filtered Water | 50 wt % or more |
| Fractionated Manuka Honey | 25 wt % or less |
| Vegetable Glycerin | 5 wt % or less |
| Pharmaceutical Grade Salt | 1 wt % or less |
| Acid Mixture | 1 wt % or less |

### Example 3

| | EFFECTIVE AMOUNT (weight % of |
|---|---|
| INGREDIENT | the composition) |
| Filtered Water | 50 wt % or more |
| Fractionated Manuka Honey | 50 wt % or less |
| Vegetable Glycerin | 5 wt % or less |
| Pharmaceutical Grade Salt | 1 wt % or less |
| Acid Mixture | 1 wt % or less |

### Example 4

| | EFFECTIVE AMOUNT (weight % of |
|---|---|
| INGREDIENT | the composition) |
| Filtered Water | 20 wt % or more |
| Fractionated Manuka Honey | 75 wt % or less |
| Vegetable Glycerin | 5 wt % or less |
| Pharmaceutical Grade Salt | 1 wt % or less |
| Acid Mixture | 1 wt % or less |

### Example 5

| | EFFECTIVE AMOUNT (weight % of |
|---|---|
| INGREDIENT | the composition) |
| Filtered Water | 10 wt % or more |
| Fractionated Manuka Honey | 80 wt % or more |
| Vegetable Glycerin | 5 wt % or less |
| Pharmaceutical Grade Salt | 1 wt % or less |
| Acid Mixture | 2 wt % or less |

### Example 6

| | EFFECTIVE AMOUNT (weight % of |
|---|---|
| INGREDIENT | the composition) |
| Filtered Water | 20 wt % or less |
| Fractionated Manuka Honey | 20 wt % to 80 wt % |
| Vegetable Glycerin | 5 wt % or less |
| Pharmaceutical Grade Salt | 1 wt % or less |
| Acid Mixture | 3 wt % or less |

### Example 7

| | EFFECTIVE AMOUNT (weight % of |
|---|---|
| INGREDIENT | the composition) |
| Filtered Water | 50 wt % or more |
| Fractionated Manuka Honey | 4 wt % or more |
| Vegetable Glycerin | 5 wt % or less |
| Pharmaceutical Grade Salt | 1 wt % or less |
| Acid Mixture | 1 wt % or less |

In one representative study, it was found that the fractionated honey composition disclosed herein effectively disrupted biofilms in a closed water system. It is further contemplated that the composition disclosed herein could therefore also effectively disrupt biofilms associated with other abiotic surfaces or inanimate objects, for example, surfaces of medical implants or other devices used with a human cavity and upon which a biofilm may form. One representative study evidencing the effectiveness of the composition in closed water systems will now be described by way of the following non-limiting example.

### Example 8

### Procedure

Each of the Fractionated Manuka Honey compositions or formulations in Examples 2-7 including Fractionated Manuka Honey concentrations of 25 wt %, 50 wt % and 75 wt % may be tested in a closed water system to determine the efficacy of the formulations against a mature Pseudomonas aeruginosa biofilm. The biofilm may be grown on borosilicate glass coupons in a CDC biofilm reactor according to ASTM Method E3161-18. After 48 hours of growth, the efficacy of the composition against a mature biofilm may be determined according to ASTM Method E2871-19. These methods are common and described in the “EPA Methods and Guidance for Testing the Efficacy of Antimicrobials against Biofilm Bacteria on Hard, Non-Porous Surfaces”, which can be found at https://www.epa.gov/pesticides/methods-and-guidance-testing-efficacy-antimicrobials-against-biofilm-bacteria-hard-non. The testing may include two contact times of 60 and 90 minutes. Products may be neutralized using DE neutralizing broth. The same diluent used to treat the product may also be used for the control coupons. Treatments may be at room temperature with no mixing. Confocal microscopy was performed on a single coupon from each treatment/time combination. The results may be reported as the biofilm log density for each coupon and the calculated mean log reduction in viable cells for product. It is believed that a reduction in viable biofilm will result from each concentration 25 wt %, 50 wt % and 75 wt % contact time combination. It is therefore believed that compositions and/or formulations including Fractionated Manuka Honey concentrations of 25 wt % to 75 wt %, for example, 25 wt %, 50 wt % and/or 75 wt %, as disclosed herein, can be used to successfully disrupt and/or otherwise remove biofilm contamination from closed water systems, medical implants or other abiotic surfaces and/or objects.

Representatively, the following treatment matrix illustrated in Table 1 was used.

TABLE 1

| Treatment Time | 60 minutes | 90 minutes |
|---|---|---|
| Control (no treatment) | 4 coupons | 4 coupons |
| Concentration 1 (50 wt % | 4 coupons | 4 coupons |
| fractionated honey) | | |
| Concentration 2 (75 wt % | 4 coupons | 4 coupons |
| fractionated honey) | | |

### Results

The test results are shown graphically in Table 2 and in tabular form in Table 3 as follows.

TABLE 3
Treatment Results

| Treatment | Log Density (CFU/cm²) | Log Reduction |
|---|---|---|
| Control 60 min | 8.70 | |
| Treatment 1 | 8.20 | 0.50 |
| (Concentration 1) 60 min | | |
| Treatment 2 | 8.15 | 0.55 |
| (Concentration 2) 60 min | | |
| Control 90 min | 8.61 | |
| Treatment 1 | 7.75 | 0.85 |
| (Concentration 1) 90 min | | |
| Treatment 2 | 8.29 | 0.31 |
| (Concentration 2) 90 min | | |

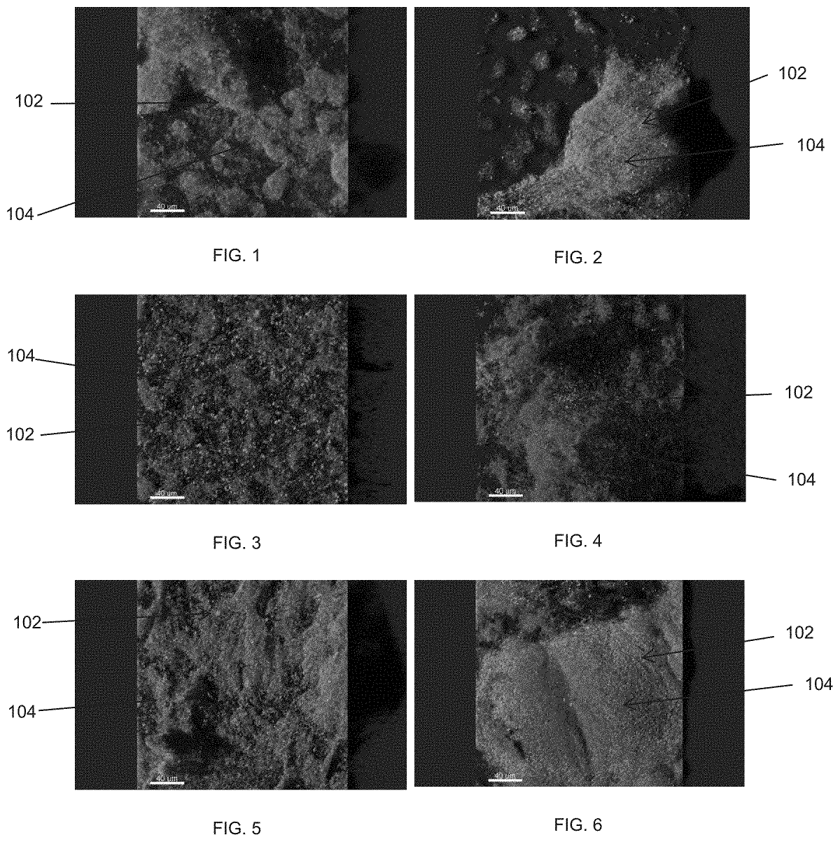

Representative confocal micrographs for each treatment and control are further illustrated in FIGS. 1-6, as will be discussed in more detail in the following Discussion of Results.

### Discussion of Results

During the study, 60 and 90 minute control coupons were colonized with a log 8.7 and log 8.61 CFU/cm2 biofilm, which is the expected level of colonization with this biofilm growth method. A maximum log reduction of 0.85 for a treatment using the compositions including the Fractionated honey concentrations of 50 wt % at the 90 minute exposure time was found. A 0.85 log reduction means that 85.9% of the cells were killed, and 14.1% remained viable. Micrographs from the experiment indicated that much of the dead biofilm cells remained on the surface after treatment with the agents tested. For imaging, the biofilms on all coupons were stained with a Live:Dead stain. This stain uses two fluorescent dyes which ostensibly differentiate between living and dead bacterial cells. Dead cells are intended to stain red while living cells are intended to stain green. The micrographs from the experiment illustrated herein as FIGS. 1-6 show mainly green cells 102 (or light gray in black and white images) in the control coupons and mainly red-stained cells 104 (or dark gray in black and white images) in the treated micrographs, suggesting many of the treated cells were not living. Representatively, FIG. 1 illustrates a 60 minute control coupon and FIG. 2 illustrates a 90 minute control coupon. It can be seen from FIGS. 1 and 2 that most of the cells are not living. On the other hand, FIG. 3 and FIG. 4 illustrate treatment 1 (Fractionated honey concentrations of 50 wt %) at 60 minutes and 90 minutes, respectively, and FIG. 5 and FIG. 6 illustrate treatment 2 (Fractionated honey concentrations of 75 wt %) at 60 minutes and 90 minutes, respectively. It can be seen from FIGS. 4-6 that most of the cells are dead following the treatments. All micrograph images are at magnification 63×.

It is further believed that a fractionated honey concentration of 15 wt %, 20 wt %, 25 wt %, and/or 30 wt % may be even more effective at disrupting biofilms. In some aspects, it is believed that other fractionated honey concentrations may result in 100% kill of a biofilm. In addition, it is believed that a reduction in viable biofilm will result from each concentration of the formulation contact time combination. It is therefore believed that compositions and/or formulations including fractionated honey concentrations of 15 wt % to 75 wt %, for example, 25 wt %, 50 wt % and/or 75 wt %, as disclosed herein, can be used to successfully disrupt and/or otherwise disrupt biofilms on abiotic or biotic surfaces.

In addition, in another study, it was found that the fractionated honey composition disclosed herein exhibited virucidal activity when tested against SARS-CoV-2. One representative study evidencing virucidal activity will now be described by way of the following non-limiting example.

### Example 9

### Procedure

### Virus, Media, and Cells

SARS-CoV-2 virus stocks were prepared by growing virus in Vero 76 cells. Test media used was MEM supplemented with 2% FBS and 50 μg/mL gentamicin.

### Virucidal Assay

The fractionated honey composition was tested at full strength. SARS-CoV-2 virus stock was added to triplicate tubes of the sample so that there was 90% virus solution by volume and 10% prepared sample. Media only was added to one tube of each prepared concentration to serve as toxicity controls. Ethanol was tested in parallel as a positive control and water only to serve as the virus control. The compound and virus were incubated at room temperature for 1 hour. Following the contact period, the solutions were neutralized by a 1/10 dilution in test media.

### Virus Quantification

Surviving virus was quantified by standard end-point dilution assay. Neutralized samples were combined for quantification for the average of triplicate tests. Samples were serially diluted using eight 10-fold dilutions in test medium. Each dilution was added to 4 wells of a 96-well plate with 80-100% confluent Vero 76 cells. The toxicity controls were added to an additional 4 wells and 2 of these wells were infected with virus to serve as neutralization controls, ensuring that residual sample in the titer assay plated did not inhibit growth and detection of surviving virus.

Plates were incubated at 37±2 degrees C. with 5% CO2. On day 6 after infection plates were scored for presence or absence of viral cytopathic effect (CPE). The Reed-Muench method was used to determine end-point titers (50% cell culture infectious dose, CCID50) of the samples, and the log reduction value (LRV) of the compound compared to the negative (water) control was calculated.

### Controls

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

### Results

Virus titer and log reduction value (LRV) for samples tested against SARS-CoV-2 are shown in Table 1. The average virus control titer was 5.0 log CCID50 per 0.1 mL and this was used for comparison of all test sample titers to determine LRV. Samples with <1 log reduction are not considered active for virucidal activity.

The limit of detection of virus was 0.7 log CCID50 per 0.1 mL. The fractionated honey composition exhibited virucidal activity when tested against SARS-CoV-2, reducing virus titer by 1.0 logs (90%), though not below the limit of detection of the assay.

Neutralization controls demonstrated that residual sample did not inhibit virus growth and detection in the endpoint titer assays in wells that did not have cytotoxicity. Positive controls performed as expected.

TABLE 1
Virucidal efficacy of Manuka honey sinus cleanser against SARS-Cov-2
after incubation with virus at 22 ± 2° C.

| | | Contact | | Neut. | Virus | VC | |
|---|---|---|---|---|---|---|---|
| Compound | Concentration | Time | Toxicityª | Control^{b} | Titer^{c} | Titer^{c} | LRV^{d} |
| Manuka honey sinus cleanser | 100% | 1-hour | None | None | 4.0 | 5.0 | 1.0 |
| Ethanol | 70% | 1-hour | None | None | <0.7 | 5.0 | >4.3 |

ªCytotoxocity indicates the highest dilution of the endpoint titer where full (80-100%) cytotoxicity was observed
^{b}Neutralization control indicates the highest dilution of the endpoint titer where compound inhibited virus CPE in wells after neutralization (Ignored for calculation of virus titer and LRV)
^{c}Virus titer of test sample or virus control (VC) in log₁₀ CCID₅₀ of virus per 0.1 mL
^{d}LRV (log reduction value) is the reduction of virus in test sample compared to the virus control

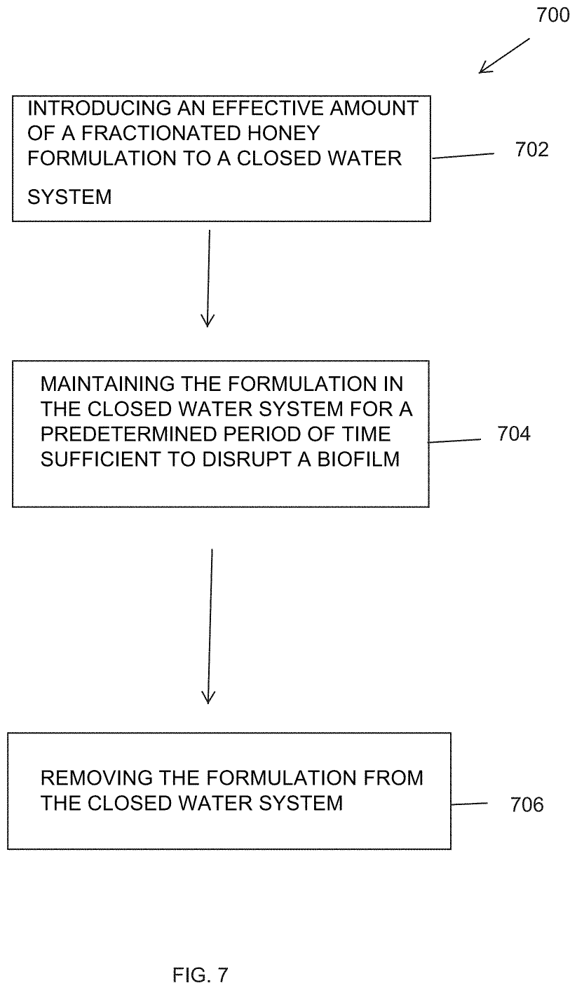

In one embodiment, the composition is used to disrupt a biofilm in a closed water system and/or otherwise associated with an inanimate object. For FIG. 7 illustrates one representative process flow 700 for disrupting a biofilm in a closed water system. In one aspect, process 700 may include introducing an effective amount of the composition in liquid form into a metal vat filled with water, and used in the closed water system, as illustrated at operation 702. In some aspects, the composition may include fractionated honey in an amount of from 15 wt % to 75 wt %, for example a formulation as disclosed in Examples 1-7, as previously discussed. In some aspects, the formulation may be maintained or otherwise remain in the closed water system for a predetermined period of time, as illustrated in operation 704. For example, in some aspects, the predetermined period of time may be about 60 minutes, about 90 minutes, from about 80 minutes to 100 minutes, about 120 minutes, more than 120 minutes, or in some aspects, the period of time may be hours or even days. In addition, in some aspects, the composition or formulation may be applied in intervals or in multiple doses for 60 minutes or more, or any amount of time sufficient to disrupt the biofilm. After a predetermined period of time found suitable for sufficiently disrupting or otherwise treating the biofilm contamination, the composition along with contaminants may be flushed from the system, as illustrated at operation 706. In another aspect, the composition may be used to disrupt a biofilm on a medical device by spraying, or otherwise applying the composition to the device or surface to be treated. In some aspects, a single dose may be applied, however, a multi-dose or interval application is also contemplated. For example, in the case of a multi-dose application, a catheter spray for longer term use could be used.

In another embodiment, the composition is administered by any technique suitable for introducing the composition in a form suitable for treatment of the desired condition, infection, or the like. Representatively, the composition may be administered via an intranasal, oral, or dermal administration route when applied or otherwise delivered to a human. For example, in some aspects, the composition may be administered to the nasal and sinus passages using a nasal spray pump with the capacity to spray the total formulation in various strengths or percentages of honey, into the furthest sinus pockets. In other aspects, the formulation may be administered to a wound, inside or on the body, using, for example, a medical device implant and/or a non-aerosol spray can, where the pressure is provided via an air bag within the can while keeping the composition isolated. In still further aspects, the composition may be delivered to the eye using a squeeze bottle for ophthalmological ingredients or an eye dropper and bottle for ophthalmological ingredients. In still further aspects, the composition may be administered to the lungs by way of a nebulizer or ventilator. In other aspects, the composition may be administered to the throat as an oral spray by mouth, for example, to the back of the throat. Alternatively, the composition may be administered into a body cavity by way of, for example, a catheter or other insertable medical device using a non-aerosol can. The composition can be made stronger in application via any of the above methods. It should further be understood that the administration routes and/or mechanisms described herein are intended as examples and should not be understood as the only suitable administration routes and/or mechanisms. Rather any administration route and/or mechanism suitable for use with the composition to treat a desired condition is contemplated.

The composition may be formed by mixing the fractionated honey composition with a carrier or vehicle until a concentration of the fractionated honey effective for treating the infection is achieved. Representatively, the composition may be mixed with a carrier or vehicle such that it includes at least 10 wt % to 90 wt % fractionated honey, or from about 20 wt % to 80 wt %, or about 25 wt %, about 50 wt % or about 75 wt % fractionated honey.

The composition may be administered by any of the above routes pursuant to a regimen for administering the fractionated honey in an amount suitable for disrupting a biofilm in a closed water system or abiotic surface or object and/or treating infection. Representatively, the composition may be administered to the desired surface, object and/or the human periodically. In some embodiments the composition is administered or applied once a day. In other embodiments, the composition is administered or applied once a day or more. For example, the composition may be administered or applied multiple times a day until the biofilm is disrupted. It is contemplated that the frequency and duration of administration or application of the composition may vary depending upon the amount of treatment agent in the composition, and the desired effects.

In some embodiments, the composition is in a single unit form, for example, the form of a pill, a capsule, a tablet, a lozenge, a mist pump, or the like. The composition may further be administered in the form of a powder. In other embodiments, the composition is administered in the form of an aqueous solution. In some embodiments, the treatment agent in the form of a powder or aqueous solution may be incorporated into a candy bar, food bar, or power bar along with substances typically used in those items, such as grains, fruits, flavorings, nuts, binders, etc. In still further embodiments, the composition is administered in the form of an implant implanted within the abiotic or biotic system which releases a desired amount of the agent over time or a dressing or patch. It is contemplated that the form of the composition may vary depending upon the desired administration route.

It should be appreciated that reference throughout this specification to “one embodiment” or “an embodiment” means that a particular feature, structure or characteristic described in connection with the embodiment is included in at least one embodiment of the present invention. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the invention.

In the foregoing specification, the invention has been described with reference to specific embodiments thereof. It will, however, be evident that various modifications and changes can be made thereto without departing from the broader spirit and scope of the invention as set forth in the appended claims. The specification and drawing are, accordingly, to be regarded in an illustrative rather than a restrictive sense.

1. A method of disrupting a biofilm in a closed water system, the method comprising:
introducing to the closed water system a composition comprising a fractionated honey in an amount effective for disrupting a biofilm in the closed water system.

2. The method of claim 1 wherein the biofilm is formed on a surface within the closed water system.

3. The method of claim 1 wherein the biofilm is caused by a gram positive bacterium, a gram negative bacterium or a virus.

4. The method of claim 1 wherein the composition comprises fractionated honey in an amount of from 20% to 80% by weight of the composition.

5. The method of claim 4 wherein the composition comprises fractionated honey in an amount of 25% to 50% by weight of the composition.

6. The method of claim 4 wherein the composition comprises fractionated honey in an amount of 50% to 75% by weight of the composition.

7. The method of claim 1 wherein introducing comprises applying the composition and maintaining the composition in the closed water system for a predetermined period of time.

8. The method of claim 7 wherein the predetermined period of time comprises 60 minutes or more.

9. The method of claim 7 wherein the composition comprises fractionated honey in an amount of from 20% by weight to 30% by weight of the composition, and the predetermined period of time comprises from 80 minutes to 100 minutes.

10. The method of claim 1 further comprising:
removing the composition from the closed water system once the biofilm is disrupted.

11. The method of claim 10 wherein removing comprises flushing the composition from the closed water system.

12. A method of disrupting a biofilm caused by a bacteria or virus formed on an abiotic surface, the method comprising:
applying to an abiotic surface a composition comprising a fractionated honey produced from Leptospermum scoparium in an amount effective for disrupting a biofilm formed on the abiotic surface.

13. The method of claim 12 wherein the abiotic surface comprises a surface of a medical implant.

14. The method of claim 12 wherein the abiotic surface comprises a surface within a closed water system.

15. The method of claim 12 wherein the amount of fractionated honey is at least 20% by weight of the composition.

16. The method of claim 12 wherein the amount of fractionated honey is at least 50% by weight of the composition.

17. The method of claim 12 wherein the amount of fractionated honey is at least 75% by weight of the composition.

18. The method of claim 12 wherein the amount of fractionated honey is 80% or less by weight of the composition.

19. The method of claim 12 wherein the composition is in the form of a spray.

20. The method of claim 12 further comprising:
removing the composition from the abiotic system once the biofilm is disrupted.
